(19) United States
(12) Patent Application Publication
(10) Pub. No.: US 2019/0224335 A1
(43) Pub. Date: Jul. 25, 2019

(54) DIRECT REPROGRAMMING OF A HUMAN SOMATIC CELL TO A SELECTED (PREDETERMINED) DIFFERENTIATED CELL WITH FUNCTIONALIZED NANOPARTICLES

(71) Applicant: STEMGENICS, INC., Seattie, WA (US)

(72) Inventor: Andranik Andrew Aprikyan, Seattle, WA (US)

(73) Assignee: STEMGENICS, INC., Seattle, WA (US)

(21) Appl. No.: 16/306,769

(22) PCT Filed: Jun. 2, 2017
(86) PCT No.: PCT/US17/35823

§ 371 Date: Dec. 3, 2018

Related U.S. Application Data
(60) Provisional application No. 62/345,360, filed on Jun. 3, 2016.

(51) Int. Cl. A61K 47/69; A61K 35/30; A61K 35/33; A61K 35/34; A61K 35/39; A61K 35/407; C12Q 1/02

(52) U.S. Cl. CPC A61K 47/6929; A61K 35/30; A61K 35/33; C12Q 1/025; A61K 35/39; A61K 35/407; A61K 47/6923; A61K 35/34

(57) ABSTRACT

This disclosure relates to compositions and methods for reprogramming an initial cell (e.g., somatic cell) to generate specialized cell types of interest, such as cardiac, hepatic, blood, neuronal and other cells from human somatic cells. In some embodiments, initial (e.g., somatic) cell is a human cell thus producing human induced cell types of interest. In some embodiments, the compositions and methods incorporate nanoparticles functionalized with biologically active molecules (RNAs, proteins, peptides and other small molecules). These newly generated (i.e., “induced”) specialized cells are useful to improve organ function and/or tissue regeneration (heart, liver, etc.) and to screen drugs for functional activity.

### CROSS-REFERENCES TO RELATED APPLICATIONS

This application claims the benefit of U.S. Provisional Application No. 62/345,360, filed Jun. 3, 2016, the entire disclosure of which is hereby incorporated by reference herein.

### STATEMENT OF GOVERNMENT LICENSE RIGHTS

This invention was made with Government support under Small Business Innovation Research (SBIR) Phase I IIP-1214943 awarded by the National Science Foundation. The Government has certain rights in the invention.

### FIELD OF THE INVENTION

This disclosure relates to methods and compositions for cell reprogramming and generating various human cell types such as cardiac, hepatic, blood, neuronal and other cells from human somatic cells. These newly generated specialized cells are useful to improve organ function and/or tissue regeneration (heart, liver, etc.) and to screen drugs for functional activity.

### BACKGROUND OF THE INVENTION

The ability of cells to normally proliferate, migrate and differentiate to various cell types is critical in embryogenesis and in the function of mature cells, including but not limited to the cells of cardiovascular and/or hematopoietic systems in a variety of inherited or acquired diseases. This functional ability of stem cells and/or more differentiated specialized cell types is altered in various pathological conditions, but can be normalized upon intracellular introduction of biologically active components or, alternatively, by transdifferentiation of other cell types into the specialized cell types that require repair or functional improvement. For example, abnormal cellular functions such as impaired survival and/or differentiation of bone marrow stem/progenitor cells into neutrophils are observed in patients with cyclic or severe congenital neutropenia who may suffer from severe life-threatening infections and may evolve to develop acute myelogenous leukemia or other malignancies (Carlsson et al., Blood, 103, 3355 (2004); Carlsson et al., Haematologica, (2006)). Another example is Barth syndrome where patients may have abnormal survival of hematopoietic cells as well as impaired cardiac function called cardiomyopathy (Makaryan et al., Eur. J. Haematol., (2012)).

Other inherited diseases like Barth syndrome, a multi-system stem cell disorder induced by presumably loss-of-function mutations in the mitochondrial TAZ gene, may be associated with neutropenia (reduced levels of blood neutrophils) that may cause recurring severe and sometimes life-threatening fatal infections and/or cardiomyopathy that may lead to heart failure that could be resolved by heart transplantation.

Treatment of neutropenic patients with granulocyte colony-stimulating factor (G-CSF) induces conformational changes in the G-CSF receptor molecule located on the cell surface, which subsequently triggers a chain of intracellular events that eventually restores the production of neutrophils to near normal level and improves the quality of life of the patients (Welte and Dale, Ann. Hematol. 72, 158 (1996)). Nevertheless, patients treated with G-CSF may evolve to develop leukemia (Aprikyan et al., Exp. Hematol. 31, 372 (2003); Rosenberg et al., Br. J. Haematol. 140, 210 (2008); Newburger et al., Genes. Pediatr. Blood Cancer, 55, 314 (2010), Aprikyan and Khuchua, Br. J. Haematol. 161, 330 (2013)), which is why alternative cell therapy approaches are being explored such as bone marrow or hematopoietic stem cell transplantation for treatment of neutropenia or ex vivo generation of cardiac cells upon differentiation of human induced pluripotent stem cells followed by transplantation of the newly generated cardiac cells into the patients' heart to fight heart failure and restore or improve cardiac muscle function.

An alternative cell therapy approach includes direct reprogramming of patients' somatic cells (e.g., fibroblasts) into functional cardiomyocytes, which could support the structural integrity of cardiac muscle and normalize the function of human heart. Recently, such direct reprogramming approaches include the use of retro- or lenti-viruses (viral vectors) harboring various cardiac specific factors including but not limited to cardiac-specific transcription factors, small molecules and microRNAs. Such viral delivery of different sets of cardiac genes with or without microRNAs was effective in direct reprogramming of human fibroblasts to induced cardiomyocyte-like cells (iCM) as evidenced by induced expression of cardiac specific genes (reviewed in Doppler, et al., Int. J. Mol. Sci. 16, 17368-17393 (2015)). Nevertheless, such viral reprogramming is associated with random integration of viral DNA into the cell genome, which is known to induce various mutations, alter normal gene expression pattern in the host cells, and trigger oncogene expression, thereby leading to cancer or other detrimental consequences. Therefore, viral reprogramming is not a plausible approach for cell reprogramming and subsequent use in humans.

The intracellular events triggered by direct reprogramming can be more effectively affected and regulated upon intracellular delivery of a cocktail of different biologically active molecules (RNAs, microRNAs, proteins, peptides and other small molecules) using distinctly non-integrating functionalized nanoparticles. Although the cellular membrane serves as an active barrier preserving the cascade of intracellular events from being affected by exogenous stimuli, these bioactive functionalized nanoparticles are capable of penetrating cellular membranes to modify the cellular function, eliminate the unwanted cells when needed, and/or directly reprogram human somatic cells into other cell types of interest.

Despite the advances in the art, a need remains for an efficient approach to deliver biologically active molecules into the interior of a cell to efficiently induce reprograming of the cell while avoiding damage to the chromosomal structure. The present invention fulfills the needs of non-integrative direct reprogramming into various cell types, preservation of intact human cell genome and provides new means for further related advantages.

### SUMMARY OF THE INVENTION

The present invention in some embodiments is directed to functionalization methods of linking proteins, peptides and/or RNA molecules to biocompatible nanoparticles for modulating cellular functions and direct reprogramming of human somatic cells into functional cells of a selected (predetermined) lineage. Such functional cells can be subsequently used in research and development, drug screening and therapeutic applications to improve cellular and/or organ function in humans. Illustrative selected (predetermined) cell types include induced cardiac cells, hepatocytes, neural cells, and the like. In some embodiments, the present invention is directed to the functionalized biocompatible nanoparticles themselves.

These and other aspects of the present invention will become more readily apparent to those possessing ordinary skill in the art when reference is made to the following detailed description.

### DETAILED DESCRIPTION OF THE INVENTION

In order to deliver biologically active molecules intracellularly, the present invention provides a universal platform based on a composition including a cell membrane-penetrating nanoparticle with covalently linked biologically active molecules. To this end, presented herein is a functionalization method that ensures a covalent linkage of proteins, peptides, and/or RNA (e.g., microRNA, RNAs encoding transcription factors, siRNAs, shRNAs, and the like) molecules to nanoparticles. The modified cell-permeable nanoparticles of the present invention provide a universal mechanism for intracellular delivery of biologically active molecules for regulation and/or normalization of cellular function in general, and direct reprogramming human somatic cells into functional cells of a selected (predetermined) lineage, which can be subsequently used in research and development, drug screening and therapeutic applications to improve cellular and/or organ/tissue function in humans. Illustrative selected (predetermined) cell types include cardiac cells, hepatocytes, neural cells, and the like.

The methods disclosed herein utilize biocompatible nanoparticles, including for example, superparamagnetic iron oxide or gold nanoparticles, or polymeric nanoparticles similar to those previously described in scientific literature (Lewin et al., Nat. Biotech. 18, 410-414, (2000); Shen et al., Magn. Reson. Med. 29, 599-604 (1993); Weissleder, et al. Am. J. Roentgeneol., 152, 167-173 (1989); each reference incorporated herein by reference in its entirety). Such nanoparticles can be used, for example, in clinical settings for magnetic resonance imaging of bone marrow cells, lymph nodes, spleen and liver (see, e.g., Shen et al., Magn. Reson. Med. 29, 599 (1993); Harisinghani et al., Am. J. Roentgenol. 172, 1347 (1999); each reference incorporated herein by reference in its entirety.) For example, magnetic iron oxide nanoparticles sized less than 50 nm and containing cross-linked cell membrane-permeable TAT-derived peptide efficiently internalize into hematopoietic and neural progenitor cells in quantities of up to 30 pg of superparamagnetic iron nanoparticles per cell (Lewin et al., Nat. Biotechnol. 18, 410 (2000)). Furthermore, the nanoparticle incorporation does not affect proliferative and differentiation characteristics of human bone marrow-derived CD34+ primitive progenitor cells or the cell viability (Maite Lewin et al., Nat. Biotechnol. 18, 410 (2000)). Accordingly, the disclosed nanoparticles can be used for in vivo tracking of the labeled cells.

The labeled cells retain their differentiation capabilities and can also be detected in tissue samples using magnetic resonance imaging. Here, we present novel nanoparticle-based compositions, which are functionalized to carry various sets of RNA (e.g., e.g., microRNA, RNAs encoding transcription factors, siRNAs, shRNAs, and the like), protein, peptide and other small molecules that can serve as excellent vehicles for intracellular delivery of biologically active molecules to target intracellular events and modulate cellular function and properties for direct reprogramming of human somatic cells into various cell types of interest.

General Description of Nanoparticle-Peptide/Protein/RNA Conjugates:

Nanoparticles can be based on iron or other material with biocompatible polymer coating (e.g., dextran polysaccharide) with X/Y functional groups, to which linkers of various lengths are attached, and which, in turn, are covalently attached to proteins, RNA (e.g., microRNA, RNAs encoding transcription factors, siRNAs, shRNAs, and the like) molecules and/or peptides (or other small molecules) through their X/Y functional groups. Linker structures are well-known and can be routinely applied to the disclosed functionalized nanoparticle design. Linkers can provide conformational flexibility to the attached bioactive compound, such as protein or polynucleotide, such that it can maintain its proper three-dimensional structure and rotate to more efficiently interact and bind with its intracellular partner.

Illustrative, non-limiting examples of functional groups that can be used for crosslinking include:

—NH₂ (e.g., lysine, a —NH₂);

—SH;

—COOH;

—NH—C(NH)(NH₂);

carbohydrate;

-hydroxyl (OH); and

attachment via photochemistry of an azido group on the linker.

Illustrative, non-limiting examples of crosslinking reagents include:

SMCC [succinimidyl 4-(N-maleimido-methyl) cyclohexane-1-carboxylate], including sulfo-SMCC, which is the sulfosuccinimidyl derivative for crosslinking amino and thiol groups;

LC-SMCC (Long chain SMCC), including sulfo-LC-SMCC;

SPDP [N-Succinimidyl-3-(pypridyldithio)-proprionate], including sulfo-SPDP, which reacts with amines and provides thiol groups;

LC-SPDP (Long chain SPDP), including sulfo-LC-SPDP;

EDC [1-Ethyl Hydrocholride-3-(3-Dimethylaminopropyl)carbodiimide], which is a reagent used to link a —COOH group with a —NH₂ group;

SM(PEG)n, where n=1, 2, 3, 4 . . . 24 glycol units, including the sulfo-SM(PEG)n derivative;

SPDP(PEG)n where n=1, 2, 3, 4 . . . 12 glycol units, including the sulfo-SPDP(PEG)n derivative;

PEG molecule containing both carboxyl and amine groups; and

PEG molecule containing both carboxyl and sulfhydryl groups.

Illustrative, non-limiting examples of capping and blocking reagents include:

citraconic anhydride, which is specific for NH;

ethyl maleimide, which is specific for SH; and

mercaptoethanol, which is specific for maleimide.

The nanoparticles useful for such purposes can contain a metal core such as iron oxide or gold, or can be polymeric nanoparticles without a metal core but containing trapped inside bioactive molecules that are released over time, leading to long-lasting effects.

In view of the foregoing, we have treated biocompatible nanoparticles with functional amines on the surface to chemically bind proteins, nucleic acids and short peptides, as described in U.S. 2014/0342004, incorporated herein by reference in its entirety. Briefly, the superparamagnetic or alternative nanoparticles can be less than 50 nm or larger in size and 10¹⁵-10²⁰ nanoparticles per ml with 10 or more amine groups per nanoparticle.

SMCC (such as from ThermoFisher) can be dissolved in dimethylformamide (DMF) obtained from, for example, ACROS (sealed vial and anhydrous) at the 1 mg/ml concentration. Sample is sealed and used almost immediately.

Ten (10) microliters of the solution are added to nanoparticles in 200 microliter volume. This provided a large excess of SMCC to the available amine groups present, and the reaction is allowed to proceed for approximately 1-2 hours. Excess SM and DMF can be removed using a centrifugal filter column (such as from Amicon) with a cutoff of 3,000 daltons. Five exchanges of volume are generally required to ensure proper buffer exchange. It is important that excess of SMCC be removed at this stage.

Any RNA or peptide based molecule, for example commercially available Green Fluorescent Protein (GFP) or purified recombinant GFP, or any other proteins of interest, can be added to the activated nanoparticles. The bioactive molecule-nanoparticle solutions are reacted and the unreacted molecules are removed by centrifugal filter units with appropriate MW cutoff (in the example with GFP it is 50,000 dalton cut-off or larger). The sample is stored at −80° C. freezer or at 4° C. Instead of using Amicon centrifugal filter columns, small spin columns containing solid size filtering components, such as Bio Rad P size exclusion columns can also be used. It should also be noted that SMCC also can be purchased as a sulfo derivative (Sulfo-SMCC), making it more water soluble. DMSO (dimethyl sufloxide) may also be substituted for DMF as the solvent carrier for the labeling reagent; again, it should be anhydrous.

All the other crosslinking reagents can be applied in a similar fashion. SPDP is also applied to the protein/applicable peptide in the same manner as SMCC. It is readily soluble in DMF. The dithiol is severed by a reaction with DTT for an hour or more. After removal of byproducts and unreacted material, it is purified by use of an Amicon centrifugal filter column with 3,000 MW cutoff.

Another means of labeling a nanoparticle with a peptide, RNA (e.g., microRNA, RNAs encoding transcription factors, siRNAs, shRNAs, and the like), or protein molecules would be to use two different bifunctional coupling reagents, as we described in US 2014/0342004, incorporated herein by reference in its entirety.

Attachment of Peptides, RNAs (e.g., microRNA, RNAs encoding transcription factors, siRNAs, shRNAs, and the like) and Proteins on a Nanoparticle. In one embodiment, various ratios of SMCC labeled proteins and peptides are added to the beads and allowed to react. Exemplary proteins and peptides are described in more detail below.

In another aspect, the present invention is also directed to a method of delivering bioactive molecules attached to functionalized nanoparticles for modulation of intracellular activity aimed at direct reprogramming of human somatic cells into other cell types (such as, e.g., iCM). For example, human cells, fibroblasts or other cell types that are either commercially available or obtained using standard or modified experimental procedures are first plated under sterile conditions on a solid surface with or without a substrate to which the cells adhere (feeder cells, gelatin, martigel, fibronectin, laminin and the like). The plated cells are cultured for a time with a specific factor combination that allows cell division/proliferation or maintenance of acceptable cell viability. Examples are serum and/or various growth factors/cytokines as appropriate for the cell-type, which can later be withdrawn or refreshed and the cultures continued. The plated cells are cultured in the presence of functionalized biocompatible cell-permeable nanoparticles with covalently linked cell-specific reprogramming factors (reprograming factors specific for the cell type of interest, such as for example, cardiac-, hepatocyte-, and neural-specific reprograming factors) attached using various methods briefly described herein and elsewhere (see, e.g., US 2014/0342004, incorporated herein by reference in its entirety) in the presence or absence of magnetic field. The use of a magnet in case of superparamagnetic nanoparticles renders an important increase in the contact surface area between the cells and nanoparticles and thereby reinforces further improved penetration of nanoparticles functionalized with peptide, protein or RNA molecules through the cell membrane. When necessary, the cell population is treated repeatedly with the functionalized nanoparticles to deliver the bioactive molecules intracellularly.

The cells are maintained attached or suspended in culture medium, and non-incorporated nanoparticles can be removed by centrifugation or cell separation, leaving cells that are present as clusters. The cells are then resuspended and recultured in fresh medium for a suitable period. The cells can be taken through multiple cycles of separating, resuspending, and reculturing, until a consequent direct reprogramming effect triggered by the specific bioactive molecules linked to the functionalized nanoparticles is observed. The current invention is applicable not only to direct reprogramming of one type of cells into another, but also as new means to control or regulate the cell fate with preservation of the original cell type. A broad range of cell types can be used such as human fibroblasts, blood cells, epithelial cells, mesenchymal cells, and the like.

Cell reprogramming, whether direct or indirect, is based on the treatment of various cell types or tissues with bioactive molecules that can include various proteins, peptides, small molecules, RNA (e.g., microRNA, RNAs encoding transcription factors, siRNAs, shRNAs), and the like. Such bioactive molecules do not penetrate through cell membrane efficiently, or at all, and may not reach the cell nuclei without a special delivery vehicle and/or specialized experimental conditions. Furthermore, these bioactive molecules have short half-life and can undergo degradation upon exposure to various proteases and nucleases. These disadvantages result in reduced efficacy of the bioactive molecules and require much higher or repeated doses of a treatment to achieve a noticeable cell reprogramming effects, if any. Therefore, in the current invention functionalized nanoparticles are used to overcome the abovementioned disadvantages. More specifically, these bioactive molecules when linked to the nanoparticles and compared with the original “naked” state, acquire new physical, chemical, biological functional properties, that confer cell-penetrating and cell nucleus-targeting ability, larger size and altered overall three-dimensional conformation as well as the acquired capability to regulate the expression of target genes of interest.

To date, a number of gene products and bioactive molecules have been reported to exhibit reprogramming effects, and the list continues to grow. For example, different sets of bioactive molecules and/or gene products were reported to induce direct reprogramming of human fibroblasts to cardiomyocytes. One such set represents a group of transcription factors. Another set includes some of these factors and additional genes along with microRNA molecules miR1 and miR133. Yet other sets include different combinations of bioactive molecules as reported (Fu J D, et al., Direct Reprogramming of Human Fibroblasts toward a Cardiomyocyte-like State. Stem Cell Reports, 1, 235-247 (2013); Nam Y J, et al., Reprogramming of human fibroblasts toward a cardiac fate. Proc. Natl. Acad. Sci. USA. 110, 5588-5593 (2013); Wada R, et al., Induction of human cardiomyocyte-like cells from fibroblasts by defined factors. Proc. Natl. Acad. Sci. USA. 110, 12667-12672, (2013); and Cao N, et al., Conversion of human fibroblasts into functional cardiomyocytes by small molecules. Science. 352, 1216-1220 (2016); each incorporated herein by reference in its entirety). Human fibroblasts transduced with viruses harboring these bioactive molecules have been reprogrammed directly into induced cardiomyocyte-like cells (iCM) as evidenced by presence of cardiac-specific markers absent in original fibroblasts. Yet, the resultant reprogrammed cells have a skewed gene expression pattern that is due to insertion of the viral and gene product-encoding DNA into the cell genome. Furthermore, the efficiency of such direct reprogramming is very low, which in part is due to a short half-life of these bioactive molecules. These problems are addressed by the present disclosure, which provides for the use of additional degradation-protecting compounds, such as a nanoparticle or a PEG or other compound or molecule functionalized with non-integrating peptides, proteins and RNA molecules, thereby preserving the cell genome intact. In some embodiments, the RNA molecule can be, e.g., microRNA, an RNA encoding a transcription factors, siRNA, shRNA, and the like.

In addition to direct fibroblast-to-cardiomyocyte reprogramming, direct reprogramming has been reported possible for the generation of hepatocytes and neural cells using different sets of bioactive molecules. For example, the FOXA3, HNF1A, and HNF4A genes, when expressed in human fibroblasts using lentiviral vectors, result in direct reprogramming of the cells and generation of functional hepatocytes as evidenced by the expression of hepatic genes and restoration of liver function in an animal model of acute liver failure. Similar to the virus-mediated direct cardiac reprogramming, this approach may result in detrimental consequences due to random integration of viral DNA into the human cell genome and development of cancer. The present invention overcomes this problem upon generation and use of the nanoparticles functionalized using abovementioned and/or other reprogramming factors as non-integrating molecules thereby preserving the cell genome completely intact.

Successful fibroblast-to-neural cell direct reprogramming was reported upon treatment of fetal fibroblasts with a single factor, Sox-2 (Ring et al., Cell Stem Cell, 11, 100-109, (2012); incorporated herein by reference in its entirety). The resultant newly reprogrammed cells exhibit neural cell phenotype and gene expression pattern with the ability to further differentiation to other neural cell types such as oligodendrocytes and astrocytes. More recently expression of Sox2 and Pax6 genes was reported to be effective in reprogramming human adult fibroblasts into neural cells (Connor et al., Direct Conversion of Adult Human Fibroblasts into Induced Neural Precursor Cells by Non-Viral Transfection. Protocol Exchange (2015), doi:10.1038/protex.2015.034; incorporated herein by reference in its entirety). There are various factors or their combinations that reprogrammed human somatic cells such as fibroblasts directly to neural cells (see, e.g., Son et al. Cell Stem Cell., 9, 205-218 (2011); Pfisterer et al., Proc. Natl. Acad. Sci., 108, 10343-10348 (2011); Ambasudhan et al., Cell Stem Cell., 9, 113-118, (2011); each reference incorporated herein by reference in its entirety.)

Similar to other reports on transdifferentiation, the direct reprogramming approaches indicated above are also based on the expression of gene products delivered to the cells using either lentiviral or retroviral vectors or plasmid DNA. Again, the use of DNA is prone to trigger unpredictable random insertion of nucleotides into the genomic DNA of the host cell thereby potentially leading to detrimental consequences or skewing the phenotype. However, attempts to implement cell reprogramming using reprogramming factors such as proteins fused to TAT-like peptides with cell-penetrating ability for cell reprogramming has been very inefficient compared with viral delivery of the genes of interests (Kim et al., Cell Stem Cell., 4, 472-476 (2009); Zhou et al., Cell Stem Cell., 4, 381-384 (2009); each reference incorporated herein by reference in its entirety), which is the major reason this approach was abandoned and not followed.

To date different factors or various combinations thereof have been reported as effective for direct reprogramming, and the list of potential factors with similar properties continues to grow. Table 1 below contains several illustrative and non-limiting examples of various bioactive factors or their combinations suitable for use in direct reprogramming according to the present invention:

TABLE 1
Illustrative reprogramming factors and combinations. Each
reference incorporated herein by reference in its entirety.

| | TD | |
|---|---|---|
| CELL TYPE | FACTOR | REFERENCE |
| iPSC | Oct4 | Takahashi, et al. (2007). “Induction |
| | Sox2 | of pluripotent stem cells from adult |
| | c-Myc | human fibroblasts by defined |
| | Klf4 | factors.” Cell 131, 861-872. |
| | Lin28 | Yu, et al. (2007). “Induced |
| | Nanog | pluripotent stem cell lines derived |
| | | from human somatic cells.” Science |
| | | 318, 1917-2920. |
| | Mir- | Anokye-Danso, et al. (2011). “Highly |
| | 302bcad/367 | efficient miRNA-mediated |
| | | reprogramming of mouse and human |
| | | somatic cells to pluripotency.” Cell |
| | | Stem Cell 8, 376-388. |
| | Mir-302 | Miyoshi, et al. (2011). “Reprogramming |
| | Mir-200c | of mouse and human cells to |
| | Mir-369 | pluripotency using mature |
| | | microRNAs.” Cell Stem Cell 8, |
| | | 633-638. |
| Cardiomyocyte | Tbx5 | Ieda, et al. (2010). “Direct |
| | Mef2c | reprogramming of fibroblasts into |
| | Gata-4 | functional cardiomyocytes by defined |
| | Mesp1 | factors.” Cell 142, 375-386. |
| | Mir-1-1 | Ivey, et al. (2008). “MicroRNA |
| | | regulation of cell lineages in mouse |
| | | and human embryonic stem cells.” |
| | | Cell Stem Cell. 2, 219-229. |
| | Oct4 | Efe, et al. (2011). “Conversion of |
| | Sox2 | mouse fibroblasts into |
| | Klf4 | cardiomyocytes using a direct |
| | C-Myc | reprogramming strategy.” Nat. Cell |
| | | Biol. 13, 215-222. |
| | CHIR99021 | Cao N, et al. (2016). “Conversion of |
| | A83-01 | human fibroblasts into functional |
| | BIX01294 | cardiomyocytes by small molecules.” |
| | AS8351 | Science DOI: 10.1126/science.aaf1502 |
| | SC1 | |
| | Y27632 | |
| | OAC2 | |
| | SU16F | |
| | JNJ10198409 | |
| Neuron | Brn2 | Vierbuchen, et al. (2010). “Direct |
| | Ascl1 | conversion of fibroblasts to functional |
| | Mytl1 | neurons by defined factors.” Nature |
| | Zic1 | 463, 1035-1041. |
| | Brn2 | Pang, et al. (2011). “Induction of |
| | Ascl1 | human neuronal cells by defined |
| | Mytl1 | transcription factors.” Nature 476, |
| | NeuroD1 | 220-223. |
| | Mir-9 | Yoo, et al. (2011). “MicroRNA- |
| | Mir-124 | mediated conversion of human |
| | Ascl1 | fibroblasts to neurons.” |
| | Mytl1 | Nature 476, 228-231. |
| | Ascl1 | Caiazzo, et al. (2011). “Direct |
| | Brn2 | generation of functional |
| | Mytl1 | dopaminergic neurons from mouse |
| | Lmx1a | and human fibroblasts.” Nature |
| | FoxA2 | 476, 224-227. |
| | Mytl1 | Ambasudhan, et al. (2011). “Direct |
| | Brn2 | Reprogramming of Adult Human |
| | Mir-124 | Fibroblasts to Functional Neurons |
| | | under Defined Conditions.” Cell |
| | | Stem Cell. 9, 113-118. |
| | Oct4 | Kim, et al. (2011). “Direct |
| | Sox2 | reprogramming of mouse fibroblasts |
| | Klf4 | to neural progenitors.” Proc. |
| | C-Myc | Natl. Acad. Sci. USA 108, |
| | | 7838-7843. |
| Dopaminergic | Ascl1 | Pfisterer, et al. (2011). “Direct |
| Neurons | Brn2 | conversion of human fibroblasts to |
| | Mytl1 | dopaminergic neurons.” Proc. Natl. |
| | Foxa2 | Acad. Sci. USA 108, 10343-10348. |
| | Lmx1a | |
| Motor Neurons | Lhx3 | Son, et al. (2011). “Conversion of |
| | Ascl1 | Mouse and Human Fibroblasts into |
| | Brn2 | Functional Spinal Motor Neurons.” |
| | Mytl1 | Cell Stem Cell 9, 205-218. |
| | Ngn2 | |
| | Hb9 | |
| | Isl1 | |
| | NeuroD1 | |
| Hepatocytes | Gata-4 | Huang, et al. (2011). “Induction of |
| | HNF1-alpha | functional hepatocyte-like cells from |
| | Foxa3 | mouse fibroblasts by defined factors.” |
| | | Nature 475, 386-389. |
| | HNF4-alpha | Sekiya, S. and A. Suzuki (2011). |
| | Foxa1 | “Direct conversion of mouse fibroblasts |
| | Foxa2 | to hepatocyte-like cells by defined |
| | Foxa3 | factors.” Nature 475, 390-393. |
| Beta-Cell | Ngn3 | Zhou, et al. (2008). “In vivo reprogramming |
| | Pdx1 | of adult pancreatic exocrine cells |
| | MafA | to beta-cells.” Nature 455, 627-632. |
| | VP16 | |
| Blood Progenitor | Oct4 | Szabo, et al. (2010) “Direct conversion |
| | Gata1 | of human fibroblasts to multilineage |
| | Gata2 | blood progenitors.” Nature 468, |
| | Gata3 | 521-526 |
| | Gata-4 | |
| Myocytes | MyoD | Davis, et al. (1987). “Expression of a |
| | | single transfected cDNA converts |
| | | fibroblasts to myoblasts.” Cell 51, |
| | | 987-1000. |
| | Mir-1-1 | Cordes, et al. (2009). “miR-145 and |
| | Mir-133 | miR-143 regulate smooth muscle cell |
| | Mir-143 | fate and plasticity.” Nature 460, |
| | Mir-145 | 705-710. |
| Osteoblast | Mir-2861 | Ivey and Srivastava (2011). |
| | | “MicroRNAs as regulators of |
| | | differentiation and cell fate decisions.” |
| | | Cell Stem Cell 7, 36-41. |

The current invention overcomes the insertional mutagenesis and skewing genotype/phenotype problems by using nanoparticles (whether metal-core (e.g., superparamagnetic iron-based or gold based nanoparticles) or non-cored (e.g., polymeric nanoparticles)) functionalized with any of the abovementioned or other bioactive molecules exposure to which may result in reprogramming of one type of cells into another cell type. The recited cell types, factors, and/or combinations of factors are not intended to be limiting and that additional factors and/or combinations will be newly discovered and that those combinations would work in the same way as described in the application.

One use of the invention is the screening/testing of a bioactive molecule (compound or compounds) for an effect on cell reprogramming. This involves combining the compound attached to the nanoparticle using methods disclosed herein with a cell population of interest (whether fibroblasts, blood cells, mesenchymal cells, and the like), culturing for suitable period and then determining any modulatory effect resulting from the compound(s). This includes direct cell reprogramming and generation of specialized cell types of interest, such as cardiac cells, hepatocytes (liver cells), or neural cells, examination of the cells for toxicity, metabolic change, or an effect on contractile activity and/or other function.

Another use of the invention is the formulation of specialized cells as a medicament or in a delivery device intended for treatment of a human or animal body. This enables the clinician to administer the functionalized nanoparticles in or around the damaged organ (e.g. heart, brain, or liver) tissue either from the vasculature or directly into the muscle or organ tissue, thereby allowing the specialized cells to engraft, limit the damage, and participate in regeneration/regrowth of the tissue's musculature and restoration of specialized function. Alternatively, the induced cardiac cells (iCM) or other cell types, as described herein, can be produced ex vivo with the described functionalized nanoparticles and administered thereafter into the area around diseased or damaged tissue of a subject.

Another application of the present disclosure is to generate and/or use the iCMs as described herein as a screening scaffold to test one or more candidate compositions for a therapeutic or pharmacological effect in a cardiac disease context. For example, the iCMs (or cell types of interest such as hepatocytes and neural cells) can be generated and cultured in vitro and contacted with a candidate pharmaceutical agent and the cells can thereafter be observed for an effect. In some embodiments, an iCM or other cell type, can be generated from a somatic cell derived from a subject with a cardiac disorder or other diseases. Accordingly, the screen for pharmaceutical activity with respect to the cardiac condition can be made for the specific genetic background of the subject in need to assess the responsiveness of the subject to the pharmaceutical agent.

Unless specifically defined herein, all terms used herein have the same meaning as they would to one skilled in the art of the present invention. Practitioners are particularly directed to Sambrook J., et al., (eds.) Molecular Cloning: A Laboratory Manual, 3rd ed., Cold Spring Harbor Press, Plainsview, N.Y. (2001); and Ausubel F. M., et al., (eds.), Current Protocols in Molecular Biology, John Wiley & Sons, New York (2010), each incorporated herein by reference.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the terms “herein,” “above,” and “below,” and words of similar import, when used in this application, shall refer to this application as a whole and not to any particular portions of the application.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited is hereby specifically incorporated by reference in their entireties.

As way of further illustration and not limitation, the following Examples disclose other aspects of the present invention.

### Example 1

The non-integrating nanoparticles are functionalized with a set of cardiac-specific transcription factors (e.g., set 1 that includes Gata4, MEF2C, TBX5, MESP1, and MYOCD recently described (Nam et al., Proc. Natl. Acad. Sci. USA. 110, 5588-5593, (2010) incorporated herein by reference in its entirety). Briefly, the human somatic cells are treated with functionalized nanoparticles once or repeatedly (2 or more times), which results in delivery of cardiac-specific factors to the cytoplasm and nucleus of the treated cells. The cells are maintained in appropriate culture medium for extended period of time and the outcome of such direct reprogramming of human somatic cells into functional cardiac cells is monitored using various molecular biology, biochemistry and cell biology techniques. Specifically, expression of cardiac specific Troponin T or tropomyosin can be determined by RNA isolation followed by real time or reverse transcribed PCR, immunostaining of the cells using appropriate antibodies, or by flow cytometry analyses of the cultured cells.

### Example 2

A different set of cardiac specific factors for direct reprogramming of human somatic cells can include nanoparticles functionalized with cardiac-specific transcription factors and microRNAs. For example, set 2 containing four proteins Gata4, Hand2, TBX5, MYOCD and two microRNAs miR-1 and miR-133. This combination of bioactive molecules introduced into the cells using viral vectors is efficient in direct reprogramming of human fibroblasts with generation of functionally active and contracting cardiomyocyte-like cells (Wada et al., Proc. Natl. Acad. Sci. USA. 110, 12667-12672, (2013)). Here, the human fibroblasts are treated with nanoparticles functionalized with set 2 of recombinant proteins and microRNAs and cultured to induce generation of human iCMs. Alternative combination of these and/or other sets of cardiac-specific factors that together trigger reprogramming of human somatic cells into cardiac cells.

The preparation of these non-integrating functionalized nanoparticles does not involve any DNA molecules that could integrate into the cell genome and disrupt normal gene expression pattern. The present invention may be embodied in other specific forms without departing from the spirit or essential characteristics thereof.

The recited cell-types, factors, and/or combinations of factors are illustrative and are not intended to be limiting. Additional factors and/or combinations, including those that are newly discovered, are encompassed in this invention and will function the same way as described herein.

### Example 3

The non-integrating nanoparticles are functionalized with a set of hepatocyte-reprogramming transcription factors that includes, as an example, FOXA3, HNF1A, and HNF4A recently described (Huang et al., Cell Stem Cell., 14, 370-384, (2014), incorporated herein by reference in its entirety). Briefly, the human somatic cells are treated with functionalized nanoparticles once or repeatedly (2 or more times), which results in delivery of liver-specific factors to the cytoplasm and nucleus of the treated cells. The cells are maintained in appropriate culture medium for extended period of time and the outcome of such direct reprogramming of human somatic cells into functional liver cells is monitored using various molecular biology, biochemistry and cell biology techniques. Specifically, expression of albumin (ALB), a-1-antitrypsin (AAT) and cytochrome P450 (CYP) enzymes can be determined by RNA isolation followed by real time or reverse transcribed PCR, immunostaining of the cells using appropriate antibodies, or by flow cytometry analyses of the cultured cells. Furthermore, the functionality of the newly generated hepatocytes can also be confirmed by evaluating metabolic activity of induced CYP enzymes using liquid chromatography-tandem mass spectrometry. These type of hepatic cells, albeit reprogrammed using lentiviral vectors, show restoration of liver function in an animal model of acute liver failure (Huang et al., Cell Stem Cell., 14, 370-384 (2014)).

The recited cell-types, factors, and/or combinations of factors are illustrative and are not intended to be limiting. Additional factors and/or combinations, including those that are newly discovered, are encompassed in this invention and will function the same way as described herein.

### Example 4

The non-integrating nanoparticles are functionalized with a set of neural-reprogramming transcription factors PAX6 and/or SOX2 recently described (Connor, Protocol Exchange doi:10.1038/protex.2015.034 (2015), incorporated herein by reference in its entirety). Briefly, the human somatic cells are treated with functionalized nanoparticles once or repeatedly (2 or more times), which results in delivery of the reprogramming factors to the cytoplasm and nucleus of the treated cells. The cells are maintained in appropriate culture medium for extended period of time and the outcome of such direct reprogramming of human somatic cells into neural progenitor cells is monitored using various molecular biology, biochemistry and cell biology techniques. Specifically, expression of neuron-specific TUJ1, MAP2, or NSE phenotypic markers together with tyrosine hydroxylase (TH), vGlut1, GAD65/67 and DARPP32 in the newly generated neural cells can be determined by RNA isolation followed by real time or reverse transcribed PCR and/or immunostaining of the cells using appropriate antibodies, or by flow cytometry analyses of the cultured neural cells reprogrammed directly from human fibroblasts.

The recited cell-types, factors, and/or combinations of factors are illustrative and are not intended to be limiting. Additional factors and/or combinations, including those that are newly discovered, are encompassed in this invention and will function the same way as described herein.

### Example 5

It is well-established that people react differently to pharmaceutical drugs. These differences can manifest at the cellular level because their cells react differently to pharmaceutical drugs based on genotype or variant developmental histories of cells among individuals (Turner R M, et al., Parsing interindividual drug variability: an emerging role for systems pharmacology. Rev Syst Biol Med. 2015 7(4), 221-41, incorporated herein by reference in its entirety). Germline variants are inherited variations and are often associated with the pharmacokinetic behavior of a drug, including drug disposition and ultimately drug efficacy and/or toxicity, whereas somatic mutations are often useful in predicting the pharmacodynamic response to drugs. Pharmacoethnicity, or ethnic diversity in drug response or toxicity, is an increasingly recognized factor accounting for interindividual variations of drug response. Pharmacoethnicity is often determined by germline pharmacogenomic factors and the distribution of single nucleotide polymorphisms across various populations (Patel J N, Cancer pharmacogenomics: implications on ethnic diversity and drug response. Pharmacogenet Genomics. 2015 25(5), 223-30, incorporated herein by reference in its entirety).

Thus, a pharmaceutical screen that utilizes patient-specific cardiac cells generated upon direct reprogramming of patients' somatic cells will reflect biases that are due to the individual's unique reaction to the pharmaceutical drugs. It may be that initial drug screens may be performed with cells from one source or individual but to broaden the applicability of a drug to the general population; a much wider selection of cells from different individuals is needed. The larger the number of source individuals the greater the probability the drug is going to have uniform response in the general population. Without this wider screening effort the drug may be effective for only a percentage of the population, for example 50, 40, or 20%, with this percentage reducing the profitability of a drug. The larger the number of source individuals for generation of cardiac cells used in drug screening, the greater the percentage of people being effectively treated with a given drug.

Similarly, participants in clinical trials may be pre-qualified for a clinical trial with a cellular assay with cardiac cells produced upon direct reprogramming of somatic cells of the candidate participant. If the cells respond well to the drug being assessed in the clinical trial the individual would be included in the clinical trial. If the cells did not respond well, the individual may be excluded from the trial. With pre-validation of the participants' better outcomes of the clinical trial may be assured.

Accordingly, despite advances in the art, this disclosure provides compositions and techniques to implement comprehensive pharmaceutical screening of drugs for cardiovascular and other disorders such that the results more accurately reflect the entire target population as a whole and avoids individual response bias and to prequalify participants in clinical trials.

The foregoing embodiments are therefore to be considered illustrative rather than limiting of the invention described herein. The scope of the invention is thus indicated by the appended claims rather than by the foregoing description, and all changes that come within meaning and range of equivalency of the claims are intended to be embraced herein.

Further, illustrative, non-exclusive examples of descriptions of some methods and compositions in accordance with the scope of the present disclosure are presented in the following numbered paragraphs. The following paragraphs are not intended to be an exhaustive set of descriptions, and are not intended to define minimum or maximum scopes, or required elements or steps, of the present disclosure. Rather, they are provided as illustrative examples of selected methods and compositions that are within the scope of the present disclosure, with other descriptions of broader or narrower scopes, or combinations thereof, not specifically listed herein still being within the scope of the present disclosure.

A1. A composition to induce differentiation of a somatic cell into a specialized cell type of interest, comprising at least one specialized cell type-inducing agent conjugated to a central nanoparticle.

A2. The composition of paragraph A1, wherein the at least one specialized cell type-inducing agent is conjugated to the central nanoparticle through a first functionalized group on the nanoparticle.

A3. The composition of one of paragraphs A1 and A2, wherein the specialized cell type is a cardiomyocyte-like cell (iCM), hepatocyte, neural, beta cell, blood progenitor cell, myocyte, osteoblast, or other cell type.

A4. The composition of one of paragraphs A1-A3, wherein the at least one specialized cell type-inducing agent comprises at least one of the agents listed in Table 1, or a functional domain thereof.

A5. The composition of one of paragraphs A1-A4, wherein the at least one specialized cell type-inducing agent comprises two, three, four, five, or more of the molecules listed in Table 1, or a functional domain thereof.

A6. The composition of one of paragraphs A1-A5, wherein the at least one specialized cell type-inducing agent comprises one or more protein or RNA molecules listed in Table 1, or functional domains thereof.

A7. The composition of one of paragraphs A1-A6, wherein the specialized cell type is a cardiomyocyte-like cell (iCM) and the one or more specialized cell type-inducing agents are selected from Gata4, MEF2C, TBX5, MESP1, Hand2, MYOCD, miR-1, and miR-133.

A8. The composition of one of paragraphs A1-A7, further comprising a penetrating peptide (CPP) conjugated to the nanoparticle through a second functionalized group on the nanoparticle.

A9. The composition of one of paragraphs A1-A8, wherein the nanoparticle has a size below about 100 nm in diameter.

A10. The composition of paragraphs A1-A9, wherein the nanoparticle has a size below about 75, 50, 40, or 30 nm in diameter.

A11. The composition of one of paragraphs A1-A10, wherein the central nanoparticle comprises iron or gold molecules.

A12. The composition of one of paragraphs A1-A11, wherein the central nanoparticle comprises polymeric molecules.

A13. The composition of one of paragraphs A1-A12, wherein the nanoparticle comprises a polymer coating.

A14. The composition of one of paragraphs A8-A13, wherein the nanoparticle comprises a polymer coating and the first and/or second functional groups are attached to the polymer coating.

A15. The composition of one of paragraphs A2-A14, further comprising a first linker molecule linking the first functional group and the at least one specialized cell type inducing agent listed in Table 1.

A16. The composition of one of paragraphs A8-A15, further comprising a second linker molecule linking the second functional group and the CPP.

A17. The composition of paragraph A18, wherein the first linker molecule has a first length, wherein the second linker molecule has a second length, and wherein the second length is greater than the first length.

A18. The composition of one of paragraphs A8-17, wherein the CPP comprises at least five basic amino acids.

A19. The composition of one of paragraphs A8-18, wherein the CPP comprises about 5, 6, 7, 8, 9, 10, 11, 12, 13, 14, 15, or more basic amino acids.

A20. The composition of one of paragraphs A8-19, wherein the CPP comprises 5, 6, 7, 8, 9, 10, 11, 12, 13, 14, 15 or more contiguous basic amino acids.

B1. A cell comprising the composition of any one of paragraphs A1-A20.

B2. The cell of paragraph B1, wherein the cell is derived from a somatic cell.

B3. The cell of one of paragraphs B1 and B2, wherein the cell is derived from a fibroblast.

B4. The cell of one of paragraphs B1-B3, wherein the cell is an induced specialized cell type of interest.

B5. The cell of paragraph B4, wherein the induced specialized cell type of interest is a cardiomyocyte-like cell (iCM), hepatocyte, neural, beta cell, blood progenitor cell, myocyte, osteoblast, or other cell type.

B6. The cell of any one of paragraphs B1-B5, wherein the cell is a human cell.

C1. A method of inducing differentiation of a somatic cell into a specialized cell type of interest listed in Table 1, comprising contacting the somatic cell with a composition of any one of paragraphs A1-A20.

C2. The method of paragraph C1, wherein the induced specialized cell type of interest is a cardiomyocyte-like cell (iCM), hepatocyte, neural, beta cell, blood progenitor cell, myocyte, osteoblast, or other cell type.

C3. The method of one of paragraphs C1 and C2, wherein the somatic cell is a fibroblast.

C4. The method of one of paragraphs C1-C3, wherein the somatic cell is contacted in vitro under culture conditions sufficient to permit differentiation of the somatic cell.

C5. The method of one of paragraphs C1-C4, wherein the somatic cell is a human cell.

D1. A method of screening a candidate pharmaceutical composition in vitro for activity in an induced specialized cell type of interest, comprising:

contacting the induced specialized cell with the candidate pharmaceutical composition; and

observing the induced specialized cell for an indication of activity.

D2. The method of paragraph D1, wherein the induced specialized cell is selected from one of the cell types listed in Table 1.

D3. The method of one paragraphs D1 and D2, wherein the induced specialized cell is a cardiomyocyte-like cell (iCM), hepatocyte, neural, beta cell, blood progenitor cell, myocyte, osteoblast, or other cell type.

D4. The method of one of paragraphs D1-D3, further comprising inducing generation of the specialized cell from a somatic cell.

D5. The method of one of paragraphs D1-D4, wherein the specialized cell is induced according to the method recited in one of paragraphs C1-05.

D6. The method of one of paragraphs D4 and D5, wherein the somatic cell is obtained from a normal subject or a subject with a specific pathological condition, and the indication of activity is an indication of activity of the pharmaceutical composition for treatment of the pathological condition in the subject.

The embodiments of the invention in which an exclusive property or privilege is claimed are defined as follows:

1. A composition to induce differentiation of a somatic cell into a specialized cell type of interest, comprising at least one specialized cell type-inducing agent conjugated to a central nanoparticle.

2. The composition of claim 1, wherein the at least one specialized cell type-inducing agent is conjugated to the central nanoparticle through a first functionalized group on the nanoparticle.

3. The composition of claim 1, wherein the specialized cell type is a cardiomyocyte-like cell (iCM), hepatocyte, neural, beta cell, blood progenitor cell, myocyte, osteoblast, or other cell type.

4. The composition of claim 1, wherein the at least one specialized cell type-inducing agent comprises at least one of the agents listed in Table 1, or a functional domain thereof.

5. The composition of one of claims 1-4, wherein the at least one specialized cell type-inducing agent comprises two, three, four, five, or more of the molecules listed in Table 1, or a functional domain thereof.

6. The composition of one of claims 1-4, wherein the at least one specialized cell type-inducing agent comprises the agents listed in Table 1, or a functional domain thereof.

7. The composition of one of claims 1-4, wherein the at least one specialized cell type-inducing agent comprises one or more protein or RNA molecules listed in Table 1, or functional domains thereof.

8. The composition of one of claim 4-7, wherein the specialized cell type is a cardiomyocyte-like cell (iCM) and the one or more specialized cell type-inducing agents are selected from Gata4, MEF2C, TBX5, MESP1, Hand2, MYOCD, miR-1, and miR-133.

9. The composition of one of claims 1-8, further comprising a penetrating peptide (CPP) conjugated to the nanoparticle through a second functionalized group on the nanoparticle.

10. The composition of one of claims 1-9, wherein the nanoparticle has a size below about 100 nm in diameter.

11. The composition of claim 10, wherein the nanoparticle has a size below about 75, 50, 40, or 30 nm in diameter.

12. The composition of one of claims 1-11, wherein the central nanoparticle comprises iron or gold molecules.

13. The composition of one of claims 1-12, wherein the central nanoparticle comprises polymeric molecules.

14. The composition of one of claims 1-13, wherein the nanoparticle comprises a polymer coating.

15. The composition of one of claims 9-14, wherein the nanoparticle comprises a polymer coating and the first and/or second functional groups are attached to the polymer coating.

16. The composition of one of claims 2-15, further comprising a first linker molecule linking the first functional group and the at least one specialized cell type inducing agent listed in Table 1.

17. The composition of one of claim 9-16, further comprising a second linker molecule linking the second functional group and the CPP.

18. The composition of claim 17, wherein the first linker molecule has a first length, wherein the second linker molecule has a second length, and wherein the second length is greater than the first length.

19. The composition of one of claim 9-18, wherein the CPP comprises at least five basic amino acids.

20. The composition of claim 19, wherein the CPP comprises about 5, 6, 7, 8, 9, 10, 11, 12, 13, 14, 15, or more basic amino acids.

21. The composition of one of claims 19 and 20, wherein the CPP comprises 5, 6, 7, 8, 9, 10, 11, 12, 13, 14, 15 or more contiguous basic amino acids.

22. A cell comprising the composition of any one of claims 1-21.

23. The cell of claim 22, wherein the cell is derived from a somatic cell.

24. The cell of claim 23, wherein the cell is derived from a fibroblast.

25. The cell of claim 22, wherein the cell is an induced specialized cell type of interest.

26. The cell of claim 25, wherein the induced specialized cell type of interest is a cardiomyocyte-like cell (iCM), hepatocyte, neural, beta cell, blood progenitor cell, myocyte, osteoblast, or other cell type.

27. The cell of any one of claims 22-26, wherein the cell is a human cell.

28. A method of inducing differentiation of a somatic cell into a specialized cell type of interest listed in Table 1, comprising contacting the somatic cell with a composition of any one of claims 1-21.

29. The method of claim 28, wherein the induced specialized cell type of interest is a cardiomyocyte-like cell (iCM), hepatocyte, neural, beta cell, blood progenitor cell, myocyte, osteoblast, or other cell type.

30. The method of one of claims 28 and 29, wherein the somatic cell is a fibroblast.

31. The method of one of claims 28-30, wherein the somatic cell is contacted in vitro under culture conditions sufficient to permit differentiation of the somatic cell.

32. The method of one of claims 28-31, wherein the somatic cell is a human cell.

33. A method of screening a candidate pharmaceutical composition in vitro for activity in an induced specialized cell type of interest, comprising:
contacting the induced specialized cell with the candidate pharmaceutical composition; and
observing the induced specialized cell for an indication of activity.

34. The method of claim 33, wherein the induced specialized cell is selected from one of the cell types listed in Table 1.

35. The method of one of claims 33 and 34, wherein the induced specialized cell is a cardiomyocyte-like cell (iCM), hepatocyte, neural, beta cell, blood progenitor cell, myocyte, osteoblast, or other cell type.

36. The method of one of claims 33-35, further comprising inducing generation of the specialized cell from a somatic cell.

37. The method of one of claims 33-36, wherein the specialized cell is induced according to the method recited in one of claims 28-32.

38. The method of one of claims 36 and 37, wherein the somatic cell is obtained from a normal subject or a subject with a specific pathological condition, and the indication of activity is an indication of activity of the pharmaceutical composition for treatment of the pathological condition in the subject.
